(19) United States
(12) Patent Application Publication
(10) Pub. No.: US 2009/0081291 A1
(43) Pub. Date: Mar. 26, 2009

(54) Sustained Release Dosage Forms For Delivery of Agents to an Oral Cavity of a User

(76) Inventors: Jerry B. Gin, Sunnyvale, CA (US); Benjamin F. Ross, Santa Clara, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 11/904,420

(22) Filed: Sep. 26, 2007

(51) Int. Cl. A61K 31/135; A61K 31/00; A61K 31/185; A61K 31/34; A61K 31/40; A61K 31/415; A61K 31/44; A61K 31/555; A61K 31/60; A61K 33/32; A61K 36/534; A61K 36/54; A61K 36/61; A61K 36/752; A61K 9/22; A61P 43/00

(52) U.S. Cl. 424/468; 424/195.18; 424/641; 424/736; 424/739; 424/742; 424/747; 514/165; 514/188; 514/343; 514/397; 514/419; 514/474; 514/578; 514/646

(57) ABSTRACT

Aspects of the invention include a sustained release dosage form that can be administered to an oral cavity, e.g., the mouth. In certain embodiments, the sustained release dosage form is formulated as a lozenge or gum that may be administered to an oral cavity of a user for the purpose of dissolving over a prolonged period of time and thereby delivering an essential oil component therein. In certain embodiments, the sustained release dosage form includes a beneficial agent and, therefore, not only provides for the prolonged delivery of an essential oil component to an oral cavity, but also provides for the sustained release of a beneficial agent thereto. In certain embodiments, the sustained release dosage form includes a biocompatible, water-insoluble polymer, e.g., ethylcellulose and an essential oil component, which are combined in such a manner so as to produce a dosage form that substantially dissolves over a prolonged period of time when positioned within an aqueous environment, such as an oral cavity of a user. In certain embodiments, the sustained release dosage form may include an additional water soluble agent, such as gum arabic, which may be included so as to further provide the dosage form with a desired dissolution characteristic. In certain embodiments, the dosage form may also include a beneficial agent to be delivered to the mouth. Methods of formulating such dosage forms and administering them to an oral cavity for the treatment of an adverse condition are also provided herein.

### BACKGROUND OF THE INVENTION

Systems for the sustained release of chemical compounds have a wide range of uses. For instance, sustained release systems have been developed to provide the gradual release of a beneficial agent within an aqueous environment of a human body, such as an oral cavity. For example, sustained release systems, including flavored lozenges and gums, have been developed to masks the symptoms of halitosis and/or to deliver a pharmacologically active agent to the mouth. Such systems find particular usefulness for the delivery of pharmacologically active agents that might otherwise have an unpleasant taste if administered to the mouth on their own.

Accordingly, the use of flavored lozenges and gums in sustained release dosage forms are well known in the art. A common formulation for a sustained release dosage from includes a gum base that encases a beneficial and/or a flavoring agent. The gum base, beneficial and/or flavoring agent are formulated in such a manner that the beneficial and/or flavoring agent is gradually released into the mouth over a relatively longer period of time when compared to that provided by other modes of delivery, such as typical gums and lozenges, mouth sprays, inhalers, mouth washes, and the like. Generally, products such as chewing gum are formulated so as to release a beneficial and/or flavoring agent contained therein over a 3 to 15 minute time period. In certain embodiments, the sustained release dosage form may be formulated as a lozenge so as to effect a greater sustained release period for the delivery of a beneficial and/or flavoring agent to the mouth.

However, the problem with typical sustained release gums and lozenges is that although they are formulated to release a beneficial and/or flavoring agent to the mouth over a longer period than that of non-sustained release gums and lozenges, and/or mouth sprays, washes, tooth pastes and the like, the referenced time period is still relatively short when compared to the length of time in an hour, several hours, or even an entire day and/or night. Further, such gum and lozenge dosage forms often lose their taste and/or effectiveness long before the indicated release period, and in the instance of lozenges, may completely dissolve rapidly, and therefore no longer be present to continue the release of the beneficial and/or flavoring agent, may not dissolve at all, or may other wise break up into non-dissolvable chunks that must either be physically removed from the mouth and disposed of or swallowed, which at times may not be ideal. This is especially problematic in situations where a beneficial agent (such as an unpleasant tasting beneficial agent) is to be delivered to the mouth, wherein once the lozenge or gum loses its ability to deliver the beneficial and/or taste-masking agent to the mouth, its usefulness is depleted, thereby requiring the user to continually replace the gum or lozenges.

Accordingly, there is a need in the art for a sustained release dosage form that provides for a longer release period of a flavored and/or otherwise beneficial agent to the mouth, wherein the dosage form gradually dissolves over a prolonged period of time without substantially breaking into non-dissolvable chunks. The subject invention presented herein meets these and other needs in the art.

### SUMMARY OF THE INVENTION

Aspects of the invention include a sustained release dosage form that can be administered to an oral cavity, e.g., the mouth. In certain embodiments, the sustained release dosage form is formulated as a lozenge or gum that may be administered to an oral cavity of a user for the purpose of dissolving over a prolonged period of time and thereby delivering an essential oil component therein. In certain embodiments, the sustained release dosage form includes a beneficial agent and, therefore, not only provides for the prolonged delivery of an essential oil component to an oral cavity, but also provides for the sustained release of a beneficial agent thereto.

In certain embodiments, the sustained release dosage form includes a biocompatible, water-insoluble polymer, e.g., ethylcellulose and an essential oil component, which are combined in such a manner so as to produce a dosage form that substantially dissolves over a prolonged period of time when positioned within an aqueous environment, such as an oral cavity of a user. In certain embodiments, the sustained release dosage form may include an additional water soluble agent, such as gum arabic, which may be included so as to further provide the dosage form with a desired dissolution characteristic. In certain embodiments, the dosage form may also include a beneficial agent to be delivered to the mouth.

Specifically, in certain embodiments, the sustained release dosage form is formulated in a manner sufficient to form a matrix that includes the various components of the sustained release dosage form, such that when positioned in an oral cavity of a user the matrix slowly dissolves and thereby delivers a flavoring and/or beneficial agent thereto, over a prolonged period of time, for instance, up to about 15 minutes, up to about 30 minutes, up to about an hour, up to about 2 hours, up to about 3, hours, up to about 4 hours, up to about 5 or 6 hours or more. Methods of formulating such dosage forms and administering them to an oral cavity for the treatment of an adverse condition are also provided herein.

### BRIEF DESCRIPTION OF THE DRAWINGS

According to common practice, the various features of the drawings may not be presented to-scale. Rather, the dimensions of the various features may be arbitrarily expanded or reduced for clarity. Included in the drawings are the following figures:

FIG. 1 depicts an oral cavity of a subject wherein the subject has not brushed the facial front surface of his teeth for three days, rather the subject gargled with LISTERINE® twice daily, for three days.

FIG. 2 depicts an oral cavity of a subject wherein the subject has not brushed the facial front surface of his teeth for three days, rather the subject administered a lozenge of the subject invention three times daily, for three days.

### DEFINITIONS

Before the present invention is further described, it is to be understood that this invention is not limited to particular embodiments described, as such may of course vary. It is also to be understood that the terminology used herein is for the purpose of describing particular embodiments only, and is not intended to be limiting. Unless defined otherwise, all technical and scientific terms used herein have the same meaning as commonly understood by one skilled in the art to which this invention belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, various publications, patents and published patent applications are cited. The disclosures of these publications, patents and published patent applications referenced in this application are hereby incorporated by reference in their entirety into the present disclosure. Citation herein by the Applicant of a publication, patent, or published patent application is not an admission by the Applicant of said publication, patent, or published patent application as prior art.

It must be noted that as used herein and in the appended claims, the singular forms “a”, “and”, and “the” include plural referents unless the context clearly dictates otherwise. Thus, for example, reference to an “essential oil component” includes a plurality of such components, and reference to “the beneficial agent” includes reference to one or more beneficial agents and equivalents thereof known to those skilled in the art, and so forth. It is further noted that the claims may be drafted to exclude any optional element. As such, this statement is intended to serve as antecedent basis for use of such exclusive terminology as “solely”, “only” and the like, in connection with the recitation of claim elements, or the use of a “negative” limitation.

In this specification and in the claims that follow, reference will be made to a number of terms, which shall be defined to have the following meanings:

“Optional” or “optionally present”—as in an “optional additive” or an “optionally present additive” means that the subsequently described component (e.g., additive) may or may not be present, so that the description includes instances where the component is present and instances where it is not.

By “pharmaceutically acceptable” is meant a material that is not biologically or otherwise undesirable, e.g., the material may be incorporated into a dosage form of the invention without causing any undesirable biological effects or interacting in a deleterious manner with any of the other components of the dosage form formulation. The term “biocompatible” is used interchangeably with the term “pharmaceutically acceptable.” When the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition. Thus, for example, “treating” a patient involves prevention of an adverse condition in a susceptible individual as well as treatment of a clinically symptomatic individual by inhibiting or causing regression of the condition.

The term “beneficial agent” refers to any chemical compound, complex or composition that exhibits a desirable effect, e.g., deemed to be beneficial. For instance, in certain embodiments, a beneficial agent may be an agent the administration of which exhibits a beneficial effect, e.g., a therapeutic effect in the treatment of an adverse physiological condition. In certain embodiments, a beneficial agent is one that interacts with the other components of the dosage form so as to produce a desirable effect. For instance, a beneficial agent may be an agent that affects the dosage form in a desirable way, such as to increase its dissolution characteristics, its duration, surface characteristics, and the like. In certain embodiments, the term may also encompass an agent that interacts with a body, or a portion thereof, to produce a desired condition, for example, a lubricated condition inside the mouth of a user. With respect to pharmaceutically active agents, the term “beneficial agent” also includes pharmaceutically acceptable derivatives of those beneficial agents specifically mentioned herein, including, but not limited to, salts, esters, amides, prodrugs, active metabolites, isomers, analogs, and the like. In certain embodiments, when the term “beneficial agent” is used, or when a particular beneficial agent is specifically identified, it is to be understood that pharmaceutically acceptable, pharmacologically active salts, esters, amides, prodrugs, active metabolites, isomers, analogs, etc. of the beneficial agent are intended as well as the beneficial agent per se. However, it is also to be understood that in certain embodiments, a beneficial agent need not be a pharmaceutically active agent nor need it have a pharmaceutical effect so long as the effect it does have is deemed beneficial.

By an “effective” amount or a “therapeutically effective amount” of a beneficial agent is meant a nontoxic but sufficient amount of the agent to provide the desired effect. The amount of beneficial agent that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0.

The term “water-insoluble” refers to a compound or composition whose solubility in water is less than 5 wt. %, for instance, less than 3 wt. %, such as less than 1 wt. %, while the term “water-soluble” refers to a compound or composition whose solubility in water is greater than or equal to 5 wt. %, for instance, greater than 10 wt. %, such as greater than 15 wt. % (measured in water at 20° C.).

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DETAILED DESCRIPTION

Aspects of the invention include a sustained release dosage form that can be administered to an oral cavity, e.g., the mouth. In certain embodiments, the sustained release dosage form is formulated as a lozenge or gum that may be administered to an oral cavity of a user for the purpose of dissolving over a prolonged period of time and thereby delivering an essential oil component therein. In certain embodiments, the sustained release dosage form not only provides for the prolonged delivery of an essential oil component to an oral cavity, but also provides for the sustained release of a beneficial agent thereto.

Accordingly, in certain embodiments, the sustained release dosage form includes a biocompatible, water-insoluble polymer, e.g., ethylcellulose, and an essential oil component, which are combined in such a manner so as to produce a dosage form that substantially dissolves over a prolonged period of time when positioned within an aqueous environment, such as an oral cavity of a user. In certain embodiments, the sustained release dosage form may include an additional water soluble agent, such as gum arabic, which may be included so as to further provide the dosage form with a desired dissolution characteristic. In certain embodiments, the dosage form also includes a beneficial agent.

Specifically, in certain embodiments, the sustained release dosage form is formulated in a manner sufficient to form a matrix that includes the various components of the sustained release dosage form, such that when positioned in an oral cavity of a user the matrix slowly dissolves and thereby delivers a flavoring and/or beneficial agent thereto, over a prolonged period of time, for instance, up to about 15 minutes, up to about 30 minutes, up to about an hour, up to about 2 hours, up to about 3, hours, up to about 4 hours, up to about 5 or 6 hours or more. Methods of formulating such dosage forms and administering them to an oral cavity for the treatment of an adverse condition are also provided herein.

The subject sustained release dosage forms of the invention will be described first, followed by a description of their use for the treatment of an adverse condition. Methods for manufacturing the sustained release dosage forms of the subject invention are also provided.

Sustained Release Dosage Forms

As summarized above, the subject invention provides for a sustained release dosage form. The sustained release dosage form is formulated to be administered to an oral cavity, such as the mouth, and may further be constituted so as to provide a flavoring and/or a beneficial agent to the oral cavity. In certain embodiments, the sustained release dosage form is formulated for the sustained release of an essential oil component and/or a beneficial agent to the mouth over a prolonged period of time.

In certain embodiments, a prolonged period of time may be a period up to about 15 minutes or up to about 6 hours or more, for instance, up to about 30 minutes to up to about 5 hours, such as up to about an hour or two to up to about 3 hours or about 4 hours. Accordingly, in certain embodiments, the sustained release dosage form of the present invention is formulated in such a manner that it slowly dissolves and may therefore its individual components may be absorbed and/or swallowed over a prolonged period of time, over which prolonged period of time a flavoring and/or beneficial agent is gradually and steadily released therein as the dosage form dissolves.

### The Biocompatible, Water Insoluble Polymer

In certain embodiments, the sustained release dosage form of the subject invention includes a biocompatible polymer. Any suitable polymer that is biocompatible and capable of forming a gradually dissolving, sustained release matrix when combined with the other components of the subject invention may be used. By “biocompatible” is meant that administration of the polymer to an oral cavity does not elicit an undesirable biological effect or produce an adverse interaction within the body and/or with any other constituent within the dosage form. In certain embodiments, the biocompatible polymer is water-insoluble. By “water-insoluble” is meant that, in certain embodiments, the polymer has a solubility in water that is less than 5 wt. %, for instance, less than 3 wt. %, such as less than 1 wt. %. In certain embodiments, the biocompatible, water-insoluble polymer may be hydrophobic. In certain embodiments, the biocompatible water-insoluble polymer may be hydrophilic.

A suitable polymer and a suitable amount of the polymer may be selected based, in part, upon its molecular weight and/or viscosity, so as to produce an overall dosage form composition with desired characteristics. For instance, a suitable polymer and amount may be one that is chosen to impart a certain property to the overall dosage form, such as to affect the dissolution characteristics of the overall dosage form in an oral cavity of a user.

Specifically, in certain embodiments, the viscosity and/or molecular weight and/or amount of the polymer may be selected, in conjunction with the other components of the dosage form, so as to be incorporated in an overall composition that is configured to produce a dosage form that gradually dissolves over a prolonged period of time in an oral cavity. For example, in certain embodiments, the biocompatible polymer and its amount are selected such that the polymer forms a matrix with the other components of the dosage form, e.g., an essential oil and/or beneficial agent, which matrix when placed in an aqueous environment, such as the mouth, may be gradually and substantially dissolved thereby slowly releasing the polymer, essential oil, and/or other components of the matrix into the mouth.

Accordingly, what is meant by “dissolve” is that the moisture of the aqueous environment may be absorbed within the matrix, which absorption causes the components of the matrix to slowly separate from the matrix and/or each other such that the individual components may be released and absorbed while the overall matrix slowly becomes smaller and smaller until the entire dosage form is substantially dissolved and its various components are absorbed and/or swallowed and/or the like. By “substantial,” in the context set forth above, is meant that an extensive amount of the dosage form dissolves, for instance, a large portion of the % weight of the individual components of the dosage form are released within the mouth and/or absorbed and/or swallowed by a user such that the overall weight of the dosage form, gradually over a prolonged period of time, becomes less and less, for example, the overall % weight of the dosage from may decrease gradually by about 70% or about 75%, such as about 80% or about 85%, for instance, about 90%, such as 95% or 98%, including 99% or more. This is in contrast to dosage forms wherein the matrix does not dissolve but remains largely intact, or degrades and/or erodes into smaller non-dissolved chunks or fragments of biocompatible polymer and/or essential oil and/or beneficial agent components, which components are either swallowed as un-dissolved chunks or physically removed from the oral cavity.

Specifically, in certain embodiments, where the dosage form includes a suitable biocompatible polymer, an essential oil component, and/or a beneficial agent in suitable amounts, a cohesive matrix may be formed such that when placed in an aqueous environment, moisture within the environment may slowly be absorbed into the matrix, which absorption both wets the dosage form and dissolves the cohesiveness of the matrix. In this manner, the essential oil component may be slowly released into the aqueous environment imparting a flavored taste therein and/or a beneficial agent may gradually be liberated from the matrix for prolonged and sustained administration thereof.

Accordingly, the dissolution characteristics of the dosage form may be controlled and finely tuned, based, in part, on the viscosity and/or molecular weight and/or amount of the biocompatible polymer and, in part, on its interaction with the other components of the dosage form. For instance, in certain embodiments, wherein a longer or smoother dissolution rate, e.g., a slower dissolving of the matrix, is desired, a biocompatible polymer with a relatively higher viscosity/molecular weight may be selected for use in formation of the dosage form matrix. In certain embodiments, wherein a shorter or less smooth dissolution rate, e.g., a less slow dissolving or degrading of the matrix is desired, a biocompatible polymer with a relatively lower viscosity/molecular weight may be selected to be used in the formation of the dosage form matrix. Hence, a wide range of polymers with varying degrees of viscosity and in differing amounts may be used in the formation of the matrix of the dosage form so as to fine tune the dissolution characteristics of the overall dosage form.

For instance, in certain embodiments, the biocompatible and water-insoluble polymer may have a solution viscosity in the range of about 1 to about 160 cP, such as a solution viscosity in the range of about 3 to about 120 cP, including a solution viscosity in the range of about 6 to about 110 cP, about 41 to 90 cP, or about 49 to about 85 cP. For example, in certain embodiments, the biocompatible and water-insoluble polymer may have a low, medium, medium-high, or high molecular weight/solution viscosity. Consequently, in certain embodiments, the biocompatible and water-insoluble polymer may be a low, medium, medium-high, or high molecular weight polymer.

Specifically, in certain embodiments, the biocompatible and water-insoluble polymer may have a low solution viscosity in the range of about 1 to about 22 cP, and therefore be a low molecular weight polymer. In certain embodiments, the biocompatible and water-insoluble polymer may have a medium solution viscosity in the range of about 23 to about 49 cP, and therefore be a medium molecular weight polymer. In certain embodiments, the biocompatible and water-insoluble polymer may have a medium-high solution viscosity in the range of about 50 cP to about 85 cP, and therefore be a medium-high molecular weight polymer. In certain embodiments, the biocompatible and water-insoluble polymer may have a high solution viscosity in the range of about 86 cP to about 110 cP or up to about 160 cP or more, and therefore be a high molecular weight polymer.

In certain exemplary embodiments, the biocompatible and water-insoluble polymer is a cellulosic polymer, such as ethylcellulose. Any suitable form of ethylcellulose may be used. For example, suitable ethylcellulose polymers that are available commercially include, without limitation, those that may be obtained from the Dow Chemical Company (Midland, Mich.) as ETHOCEL® ethylcellulose, e.g., ETHOCEL® Standard 4 Premium (e.g., a polymer with a solution viscosity range approximately 3 to 5.5 cP, ethoxyl content 48.0-49.5%), ETHOCEL® Standard 7 Premium (e.g., a polymer with a solution viscosity range approximately 6 to 8 cP, ethoxyl content 48.0-49.5%), ETHOCEL® Standard 10 Premium (e.g., a polymer with a solution viscosity range approximately 9 to 11 cP, ethoxyl content 48.0-49.5%), ETHOCEL® Standard 14 Premium (e.g., a polymer with a solution viscosity range approximately 12.6 to 15.4 cP, ethoxyl content 48.0-49.5%), ETHOCEL® Standard 20 Premium (e.g., a polymer with a solution viscosity range approximately 18 to 22 cP, ethoxyl content 48.0-49.5%), ETHOCEL® Standard 45 Premium (e.g., a polymer with a solution viscosity range approximately 41 to 49 cP, ethoxyl content 48.0-49.5%), ETHOCEL® Standard 100 Premium (e.g., a polymer with a solution viscosity range approximately 90 to 110 cP, ethoxyl content 48.0-49.5%), ETHOCEL® Medium 50 (e.g., a polymer with a solution viscosity range approximately 43 to 55 cP, ethoxyl content 45.0-47.0%), ETHOCEL® Medium 70 (e.g., a polymer with a solution viscosity range approximately 63 to 85 cP, ethoxyl content 45.0-47.0%), ETHOCEL® Medium 100 (e.g., a polymer with a solution viscosity range approximately 90 to 110 cP, ethoxyl content 45.0-47.0%), and ETHOCEL® HE 10 (e.g., a polymer with a solution viscosity range approximately 9 to 11 cP, ethoxyl content 49.5-52.0%), with all solution viscosities determined using an Ubbelohde viscometer and a solvent mixture of 80% toluene and 20% alcohol.

In certain embodiments, the average particle diameter of the biocompatible and water-insoluble polymer, such as ethylcellulose, may be varied so as to alter the overall characteristics of the dosage form. For instance, in certain embodiments, the particle size and/or average particle diameter of the polymer may be varied so as to control the dissolution characteristics of the overall dosage form. Specifically, in certain embodiments; such as where a more cohesive matrix is desired, the polymer for use in conjunction with the subject invention may be a micronized composition having a substantially uniform particle diameter. A more cohesive matrix provides for a dosage form that dissolves more slowly and/or evenly when compared to a dosage form that does not have a cohesive matrix. In certain embodiments, such as where a less cohesive matrix is desired, the polymer may be a more coarse composition having an average diameter particle size with a desired degree of non-uniformity. In this manner, by varying the average diameter particle size of the polymer composition to be formulated into the matrix, a final dosage form with a desired dissolution pattern may be formulated. For example, having a more uniform average particle size may lead to a dosage form with a cohesive matrix that dissolves smoothly rather than breaking up into smaller particles.

Specifically, where a smooth, more even dissolution pattern and sustained release matrix is desired, a more cohesive matrix including a micronized polymer composition with a substantially uniform average particle size/diameter may be formulated. Accordingly, in certain embodiments, the water-insoluble polymer comprises a monodisperse population of particles. The term “monodisperse” refers to a population of particles (e.g., a colloidal system of water-insoluble polymer particles) wherein the particles have substantially identical size and shape. For the purpose of the present invention, a “monodisperse” population of particles means that at least about 60% of the particles, such as at least about 75-90% of the particles, for instance, at least about 90% or more, fall within a specified particle size range. A population of monodisperse particles deviates less than 10% rms (root-mean-square) in diameter, for instance, less than 5% rms. Hence, in certain embodiments, the f water-insoluble polymer comprises a population of polymer particles that are identically sized. By “identically sized” is meant that the particles have essentially the same diameter.

For instance, in certain embodiments, such as where it may be important to admix one or more polymers for the production of a dosage form of the subject invention and/or to reach steady state faster, the particle sizes of the one or more (e.g., two different) polymers may be substantially identical in size. Specifically, if two different polymers are to be mixed in the formation of the dosage form, wherein the polymers have substantially the same relative density, the particle sizes and/or average particle diameter may be approximately identical, and/or the individual particle size distribution may be narrow.

Additionally, where a smooth, more even dissolution pattern and sustained release matrix is desired, a more cohesive matrix including a micronized polymer composition with a substantially uniform average particle size/diameter may be formulated. For instance, in certain embodiments, a suitable biocompatible, water-insoluble polymer composition having a substantially uniform average particle diameter may be used to form a matrix that gradually and slowly dissolves over a prolonged period of time such that the overall dosage form may substantially evenly dissolve and be gradually absorbed or swallowed within the mouth. In such an embodiment, the components of dosage form are selected and formulated to produce an overall dosage form that substantially dissolves and does not substantially break up into a plurality of chunks. For example, in certain embodiments, the components of dosage form are selected and formulated to produce an overall dosage form that not only dissolves but entirely or substantially entirely dissolves.

Accordingly, a suitable average particle size and diameter for a biocompatible and water-insoluble polymer that may be useful in conjunction with the subject invention may be a micronized polymer, e.g., micronized ethylcellulose polymer, wherein the average particle diameter is in the range of about 1 micron to about 250 microns, for instance, about 10 microns to about 100 microns, including about 20 microns to about 50 microns, such as 25 microns. In certain embodiments, a micronized polymer suitable for use with the present invention may have an average particle diameter of less than 75 microns, with a mean of about 20 microns, or a average particle diameter of less than 50 microns, with a mean of about 10 microns.

For instance, in certain embodiments, particles of a suitable water-insoluble polymer may be micronized and/or passed through a mesh screen, such as a 20, 40, 80, or more mesh screen to produce a composition of various particle sizes. For example, in certain embodiments, where it may be desired to have a dosage form with a more rapid dissolution rate (e.g., about an hour), a polymer of the subject invention may be passed through a bigger mesh screen to produce polymer particles wherein the average particle size is, for example, about 170 or more microns. Where it may be desired to have a dosage form with a longer dissolution rate (e.g., about two hours or more), a polymer of the subject invention may be passed through a smaller mesh screen to produce polymer particles wherein the average particle size is, for example, about 80 or less microns, such as between 30 to 60 microns.

In certain embodiments, the biocompatible and water-insoluble polymer may be a lactic acid polymer. A suitable lactic acid polymer may be a homopolymer or a copolymer. For instance, a suitable lactic acid copolymer may be a copolymer of lactic acid with glycolic acid, also termed “poly(lactide-co-glycolide.” The lactic acid polymer may be in enantiomerically pure form, as D-lactic acid or L-lactic acid, or it may be in the form of a racemic mixture of the two enantiomers. Accordingly, suitable lactic acid polymers include poly(D,L-lactic acid), poly(D-lactic acid), poly(L-lactic acid), poly(D,L-lactide-co-glycolide), poly(D-lactide-co-glycolide), and poly(L-lactide-co-glycolide). Where a poly(lactide-co-glycolide) polymer is provided, the amount of glycolic acid in the copolymer may be 50 mole % or less. Additionally, any poly(lactide-co-glycolide) selected as the polymer may contain about 1 mole % to about 50 mole %, such as about 15 mole % to about 50 mole %, including about 15 mole % to about 35 mole %, glycolic acid. Hence, the cellulosic polymer can be any such polymer capable of rendering the lactic acid polymer suitable for sustained release in the context of the subject invention. Additionally, suitable lactic acid polymers and copolymers may have an average molecular weight in the range of about 10,000 to 125,000.

In certain embodiments, release rate modifiers or accelerators (e.g., elements that directly or indirectly advance the dissolution of the matrix) may be used, for instance, in order to adjust the duration of the time period over which the flavoring agent and optionally other agent(s) are released. Suitable release rate modifiers that may function by advancing the dissolution of the matrix, thereby directly or indirectly aiding the release of the components of the matrix into the oral cavity, may include water-soluble cellulosic polymers such as methylcellulose (MC), hydroxypropyl cellulose (HPC), and hydroxypropyl methylcellulose (HPMC). Ingestible organic solvents, such as ethyl acetate and ethanol, may also be included. The weight ratio of release rate accelerator to the polymer (e.g., lactic acid polymer) may be in the range of about 0.05:1.5 to about 0.5:1.25, including about 0.1:1.1 to about 0.5:1.

### The Essential Oil Component

In certain embodiments, the sustained release dosage form of the subject invention includes an essential oil component. An essential oil component may include any suitable essential oil, an essential oil constituent, and a mixture thereof. A wide range of essential oil components are well known and available in the art. These essential oil components may be used individually or may be combined with one, two, three, or more additional essential oil components to produce a particular flavor mix. Hence, an essential oil component of the subject invention may include one or more essential oil components (e.g., a mixture of such components).

A suitable essential oil component may be one such that upon admixture with the biocompatible, water-insoluble polymer and/or beneficial agent results in a matrix that when administered to an oral cavity (e.g., the mouth), gradually dissolves thereby releasing the essential oil component (as well as any other incorporated component) into the oral cavity over a prolonged period of time. A suitable essential oil component may be a pharmaceutically acceptable essential oil and/or a chemical constituent thereof that has been selected to impart a desired flavor to an oral cavity, e.g., for the purpose of providing a pleasant taste or smell in the mouth or masking a unpleasant taste or smell already therein. In certain embodiments, although the essential oil component may be pharmaceutically acceptable, the essential oil component is not pharmaceutically active.

Accordingly, a suitable essential oil may be a naturally occurring compound or composition that accumulates in the oil cells, glandular trichomes, and oil or resin ducts of aromatic plants. For instance, a suitable essential oil that may be included in a dosage form of the subject invention may be one or more of: a citrus oil; such as lemon oil, lime oil, neroli oil, and orange oil; a mint oil, such as peppermint oil and spearmint oil; and other oils such as anise oil, cardamom oil, cinnamon oil, clove oil, coriander oil, eriodictyon fluidextract, eucalyptus oil, fennel oil, glycyrrhiza extract, lemongrass oil, and nutmeg oil.

Additionally, as is widely known in the art, essential oils may contain a number of other constituents that may by themselves be included in a dosage form of the subject invention. For instance, a suitable essential oil constituent may be hydrocarbon containing constituent, such as a terpene and/or a sesquiterpene. The term “Terpene,” as used herein, generally refers to hydrocarbons of the formula C10H16, and, as the term is used herein, may also encompass terpene analogs of the formula CnH2n-4, as well as terpenes and terpene analogs substituted with one or more nonhydrogen substituents and/or containing a heteroatom such as N, O, or S. Analogously, “sesquiterpenes,” as used herein, generally refers to hydrocarbons of the formula C15H24, and may also encompass sesquiterpene analogs of the formula CnH2n-6 as well as substituted and/or heteroatom-containing derivatives thereof.

It will be appreciated from the foregoing definitions that terpenes and sesquiterpenes can have any number of molecular structures, including acyclic, monocyclic, bicyclic, and polycyclic structures, wherein the bicyclic and polycyclic structures may or may not be “bridged” bicyclic and polycyclic compounds. In general, however, the terpenes that are more commonly used as flavoring agents contain two double bonds and one cyclic group (e.g., β-phellandrene) or one double bond and two cyclic groups in a bridged bicyclic structure (e.g., β-pinene). Specific examples of terpenes and sesquiterpenes that can be advantageously used as flavoring agents herein include: the terpenes d,l-camphene, d-camphene, l-camphene, Δ3-carene, trans-β-ocimene, cis-β-ocimene, trans-α-ocimene, cis-α-ocimene, β-pinene, β-phellandrene, α-terpinene, β-terpinene, and γ-terpinene; and the sesquiterpenes α-cadinene, β-cadinene, α-caryophyllene, copaene, β-farnesene, isocaryophyllene, and ylangene.

Accordingly, any suitable essential oil component and any suitable amount of an essential oil component may be included, wherein the choice of which type and amount of essential oil to be used may depend, in part, upon both the intended flavor of the overall dosage form and/or its intended use. For instance, if the intended use of the dosage form is for the treatment (e.g., masking) of dry mouth and/or halitosis, a mint oil, such as peppermint oil and/or spearmint oil, may be used. Additionally, for example, where the dosage form is intended for use as a diet aid, a citrus oil, or other oil that may impart a flavor associated with a foodstuff, may be used, for instance, so as to help satisfy a need for the taste of food in the mouth.

The amount of the essential oil component to be included may be readily chosen and empirically determined so as to not only produce a desired effect (e.g., taste, smell, etc.) in the mouth, but also a desired characteristic of the overall dosage form. For instance, the amount of the essential oil component to be included may be varied in order to regulate both the intensity of the flavor of the dosage form as well as its strength (e.g., the consistency and cohesiveness of the overall formulation). For example, both lower and higher levels of the essential oil component, relative to the polymer and/or water-soluble component, may give rise to a more flexible or brittle (respectively) matrix that more rapidly degrades or erodes, where as more equal levels of the essential oil component, relative to the polymer and/or binder, may give rise to a stronger, more cohesive matrix and thus provide for a slower release rate.

Specifically, increasing the amount of the essential oil component relative to the polymer and/or binder component may result in a less cohesive matrix (e.g., wherein the polymer becomes dissolved in the essential oil), a more rapid degradation or erosion, and, therefore, a faster release rate (e.g., of the oil and/or water-soluble component and/or beneficial agent). Likewise, a decreased amount of the essential oil component relative to the polymer and/or binder component results in a matrix that has localized hard and rigid pockets of polymer that have not been associated with the essential oil component, hence, the overall dosage form may be less cohesive and more easily destabilized (e.g., because of a lack of essential oil component, which in some instances, may act as a glue holding the matrix together), again resulting in a more rapid degradation or erosion, and, therefore, a faster release rate.

Accordingly, in certain embodiments, the amount of the essential oil component may be selected such that upon admixture with the biocompatible, water-insoluble polymer and water-soluble component (if included), results in a strong, cohesive matrix that when administered to the mouth, gradually dissolves, thereby slowly releasing the essential oil component (as well as any other incorporated component) into the mouth over a prolonged period of time. Additionally, where the dosage form includes a water-soluble component and/or beneficial agent, as the matrix dissolves the essential oil component may release a fractionate amount of the water-soluble component and/or beneficial agent that may be associated with the essential oil component. Accordingly, the amount of essential oil component can be varied to affect a desired dissolution rate of the overall matrix.

### Water-Soluble Agents

In certain embodiments, the sustained release dosage form of the subject invention includes a water-soluble agent. Any suitable water-soluble agent in any suitable amount may be used so long as the water-soluble agent is capable of being combined with the water-insoluble polymer and/or essential oil component to form a dosage form which includes a matrix that when positioned in the oral cavity of a user gradually dissolves over a prolonged period of time. Specifically, in certain embodiments, a suitable water-soluble agent in a suitable amount to be used in accordance with the subject invention, may be one that is selected such that when the water-soluble agent is combined with the biocompatible, water-insoluble polymer and/or the essential oil component and/or beneficial agent, a dosage form is produced that when administered to an oral cavity (e.g., the mouth) slowly dissolves over a prolonged period of time thereby releasing the individual components of the dosage form into the mouth.

Accordingly, in certain embodiments, a suitable water-soluble agent may be gum arabic. For instance, in certain embodiments, gum arabic may be added to the matrix composition to help coalesce the overall matrix, e.g., during the formation of the dosage form and/or may contribute to the gradual and consistent dissolution profile of the overall dosage form. For example, because gum arabic is water soluble, when the dosage form is placed in an aqueous environment, the gum arabic therein may promote the absorption of moisture (e.g., saliva, water, or the like) into the matrix of the dosage form, which in turn may promote the gradual dissolution of the dosage from as the matrix slowly dissolves. Specifically, a water-soluble agent, such as gum arabic, may be included in the dosage form at a quantity and in a manner so as to promote the consistent and complete dissolution and/or absorption/consumption of the entire dosage form (e.g., the entire matrix of the dosage form dissolves).

Accordingly, in certain embodiments, a water-soluble agent, such as gum arabic, may be included in the formulation of the matrix as a fine powder. For example, a dry powder of the water-soluble agent, e.g., gum arabic, may be formed, e.g., by milling or spray drying, so as to obtain a suitable mean diameter particle size (e.g., a mean diameter particle size of about 10 microns). For example, the average diameter particle size may range from about 1 micron to about 100 microns, for instance, about 5 microns and 50 microns, such as 10 microns and 25 microns, including 15 microns. In certain embodiments, the mean diameter particle size is less than 50 microns, less than 25 microns, for instance, less than 10 microns, and in certain embodiments, the overall diameter particle size is uniform. Once obtained, the water-soluble agent, e.g., gum arabic, may be added to the overall formulation in an amount to help provide for a desired dissolution characteristic of the matrix and overall dosage form.

Hence, in certain embodiments, the water-soluble agent comprises a monodisperse population of particles. The term “monodisperse” refers to a population of particles (e.g., a colloidal system of particles) wherein the particles have substantially identical size and shape. For the purpose of the present invention, a “monodisperse” population of particles means that at least about 60% of the particles, such as at least about 75-90% of the particles, for instance, at least about 90% or more, fall within a specified particle size range. A population of monodisperse particles deviates less than 10% rms (root-mean-square) in diameter, such as less than 5% rms. Hence, in certain embodiments, the water-soluble agent comprises a population of particles that are identically sized. By “identically sized” is meant that the particles have essentially the same diameter.

Hence, in certain embodiments, the water-soluble agent, such as gum arabic, makes up from about 5% to about 50% or the overall dosage from, such as from about 10% to about 25%, including about 15% to about 20% of the dosage form. In certain embodiments, the ratio of the water-soluble agent to the polymer and essential oil is from about 0.25:1:1 to about 1:1:1, including about 0.5:1:1 to about 0.6:1:1, including about 0.75:1:1.

With respect to the overall dosage forms of the subject invention, any suitable amount of biocompatible, water-insoluble, essential oil component and/or beneficial agent may be used in the formulation of the dosage form. For instance, in certain embodiments, the overall dosage form may include from about 1% to about 25% up to about 50% or more of the water-insoluble polymer, from about 1% to about 25% up to about 50% or more of the essential oil component, and from about 1% to about 25% or up to 50%, 60% or more other excipients, sweeteners, or the like. These amounts, however, may be varied so as to produce an overall dosage form with the desired consistency, release rate, and dissolution characteristics.

For instance, in certain embodiments, the total amount of the water-insoluble polymer in the overall dosage form may range from about 5% or about 8% to about 80% or about 90%, such as about 15% to about 60%, for instance, about 25% to about 50%, including about 30% to about 40%. For example, where a longer lasting, less moisturizing, slower release rate formulation is desired, an increased amount of water-insoluble polymer relative to the other constituents of the dosage form may be used.

In certain embodiments, the total amount of the essential oil component in the overall dosage form may range from about 3% or about 5% to about 75% or about 85%, such as 10% to about 60%, for instance, 15% to about 50%, including about 25% to about 35%. For instance, where a wetter dosage form and a more rapid release rate formulation are desired a higher overall concentration of essential oil component may be used relative to the polymer.

In certain embodiments, the total amount of the water-soluble agent in the overall dosage form may range from about less than 1% or about 3% to about 75% or about 80%, such as about 5% to about 60%, for instance, about 15% to about 50%, including about 20% to about 25% or about 30%. In certain embodiments, where a longer lasting, slower release rate dosage form is desired, the % amount of water-soluble agent may be about 15% to about 20% or 25%.

Specifically, in certain embodiments, the ratio of the amount of water-insoluble polymer to essential oil component may be about 1:1, for instance, about 2:1 or greater, such as 3:1 or greater, 4:1 or greater polymer to essential oil component. In certain embodiments, the ratio of the amount of water-insoluble polymer to essential oil component may be less than about 10:1, less than about 8:1, less than about 6:1, less than about 5:1, less than about 4:1, less than 3:1, less than about 2:1. In certain embodiments, the ratio of the amount of essential oil component to water-insoluble polymer may be about 1:1 or about 1:2, for instance, 1:3, such as about 1:4, about 1:5, or about 1:8 essential oil component to water-insoluble polymer. In certain embodiments, the ratio of the amount of water-insoluble polymer to water-soluble agent (if included) may be about 1:1 or about 5:1, for instance, about 8:1, such as 10:1 polymer to water-soluble agent. In certain embodiments, the ratio of the amount of water-soluble agent (if included) to water-insoluble polymer may be about 1:1 or about 1:5, for instance, about 1:8, such as 1:10 water-soluble agent to polymer. In certain embodiments, the ratio of the amount of essential oil component to water-soluble agent (if included) may be about 1:1 or about 2:1, for instance, about 4:1, such as 8:1 essential oil component to water-soluble agent. In certain embodiments, the ratio of the amount of water-soluble agent (if included) to essential oil component may be about 1:1, for instance, about 1:1 or about 1:2, such as 1:4 or about 1:8 water-soluble agent to essential oil component.

In certain embodiments, the sustained release biocompatible, water-insoluble polymer, essential oil component, water-soluble agent, and/or beneficial agent may be incorporated into a dosage form such as a tablet, candy lozenge, a semi-liquid, semi-solid, troche, gel, semi-gel, or gum. In certain embodiments, the components of the sustained release dosage form (e.g., polymer, essential oil component and/or water-soluble agent and/or beneficial agent) are present in one or more layers, such as a plurality of layers, for instance 2, 3, or more layers. In certain embodiments, the components of the dosage form are present in separate layers, and therefore, the dosage form includes a plurality of separate layers, e.g., 2, 3, 4, or more layers. In certain embodiments, the components of the sustained release dosage form (e.g., polymer, essential oil component and/or water-soluble agent and/or beneficial agent) are not present in layers but comprise a heterogeneous mixture. For instance, in certain embodiments, the components of the dosage form (which may include one or more beneficial agents) may be formulated in conjunction with a chewing gum base, such that the overall dosage form is a chewing gum that provides for sustained release of an essential oil, beneficial agent, or the like. Accordingly, in certain embodiments, a dosage form of the subject invention is not a tablet.

Specifically, the dosage form may be formulated as a chewing gum wherein the components of the sustained release matrix are combined with a gum base, wherein the gum base represents on the order of 5 wt. % to 90 wt. %, for instance about 5 wt. % to 50 wt. % of the gum. Any conventional gum base may be used, so long as there is no deleterious interaction between the gum base and any of the other components of the dosage form, e.g., the biocompatible polymer, essential oil, or other components of the chewing gum. For instance, a suitable gum base may include, by way of example, elastomers, elastomer plasticizers, waxes, fats, oils, softeners, emulsifiers, fillers, texturizers, and miscellaneous ingredients such as preservatives, colorants, whiteners, and the like. Most gum bases will include at least one elastomer, e.g., a synthetic elastomer such as polyisobutylene, polybutadiene, isobutylene-isoprene copolymer, styrene-butadiene copolymer, polyvinyl acetate, ethylene vinyl acetate, or polyvinyl alcohol, or a natural elastomer, including natural rubbers as well as natural gums (e.g., chicle). Further, the gum may be in the form of a tablet, e.g., a tablet coated with a layer of a quickly dissolving colored or whitened film that provides a desirable appearance and smooth texture. Such film coatings may be comprised of natural and/or synthetic hydrophilic polymers such as cellulosics, polyethylene glycol, and the like.

In certain embodiments, the lozenges or gum of the subject invention are not only formulated to evoke a pleasant sensation of flavor and/or deliver a beneficial agent within the mouth, they may be configured to be comfortably retained in the mouth for an extended period of time, for instance, by having a small size and/or a soft, rubbery consistency or a pliable, sticky consistency. For instance, where a soft, rubbery, and nontacky lozenge or gum is desired, a higher molecular weight polymer may be employed in the formation of the matrix.

However, in certain embodiments, where a pliable, sticky lozenge or gum is desired a lower molecular weight water-insoluble polymer may be employed in the formation of the matrix. With a chewing gum, for example, the use of a higher molecular weight polymer in the formation of the matrix may result in a gum that lasts longer than a gum prepared with a lower molecular weight polymer but that is otherwise identical. It should be noted that using a lower molecular weight polymer may enable the incorporation of a smaller fraction of essential oil component without reducing the overall strength of the matrix.

Accordingly, by varying the molecular weight of the water-insoluble polymer, and/or by incorporating an ingestible solvent, such as ethanol or ethyl lactate, the lozenge or gum may be rendered either adhesive or nonadhesive. For instance, the use of a lower molecular weight polymer in the dosage form may give rise to a sticky, pliable lozenge or gum that can adhere to the gums, teeth, or dental appliance(s) of a user, while the use of a higher molecular weight water-insoluble polymer may give rise to a soft, rubbery lozenge that is substantially nontacky. Incorporation of an ingestible solvent such as ethanol, ethyl acetate or ethyl lactate, or the like, can further increase adhesion.

Hence, in certain embodiments, a flavored dosage form is provided for delivering an essential oil component and/or a beneficial agent to a mucosal surface within the mouth, wherein the dosage form may have at least one adhesive surface that serves to adhere the dosage form to a mucosal surface or the teeth. In such embodiments, the dosage form may include a lower molecular weight water-insoluble polymer, such as ethylcellulose, wherein the low molecular weight polymer may have a solution viscosity in the range of approximately 1 to 6 to 15 cP as determined at 25° C. using a 5 wt. % aqueous solution; the dosage form may also include an essential oil component selected from essential oils, individual terpenes, and individual sesquiterpenes; a water-soluble component, such as gum arabic; a beneficial agent; a sweetening agent, and the like. The beneficial agent may be, for example, an anti-infective agent, a local anesthetic agent, a local anti-inflammatory agent, or the like, or other beneficial agent as herein described below. The dosage form, accordingly, may be configured to release a beneficial agent to an oral cavity or a mucosal surface therein over an extended time period.

As may be surmised from the above description, the dosage forms of the invention are useful for the delivery of an essential oil component and/or beneficial agent to the teeth or a mucosal surface within the oral cavity. Delivery to a mucosal surface within the oral cavity may be used within the context of systemic drug administration, in which case the beneficial agent is actually delivered transmucosally, e.g., through the buccal mucosa of the gums.

In this embodiment, the dosage form may be composed of a matrix, as described above with regard to sustained release lozenges, but may be formulated so as to have a surface that is sufficiently tacky to enable the dosage form to adhere to the teeth or a mucosal surface within the mouth. This may be accomplished by using a relatively low molecular weight biocompatible polymer, as discussed infra, and/or by incorporating one or more adhesive polymers that are conventionally used in buccal drug delivery systems, e.g., polyisobutylene, polyisoprene, acrylic acid polymers and copolymers (e.g., those known as “carbomers,” polyalkylene oxides (e.g., polyethylene glycol and copolymers thereof), polyvinyl lactams (e.g., polyvinyl pyrrolidone), and cellulosic materials (e.g., hydroxypropylmethyl cellulose). In certain embodiments, the dosage form may be made adhesive by using a lower molecular weight water-insoluble polymer rather than by incorporation of additional polymers not contained within the matrix. When the dosage forms of the invention serve as transmucosal delivery systems, various carriers and additives may be incorporated as is well known in the art of transmucosal (e.g., buccal) drug delivery. Other additives include permeation enhancers such as polyethylene glycol esters, long-chain fatty acid esters of diols and triols (e.g., glycerol monolaurate, propylene glycol monolaurate), lower alkanols, and the like.

In addition to delivering beneficial agents to an oral cavity, it is to be noted that in certain embodiments, the sustained release of an essential oil acts as a powerful flavoring agent within the lozenge or gum, thereby providing for extremely effective taste-masking. Hence, the lozenges and gums of the subject invention can therefore be used to deliver a host of beneficial agents whose bitter or otherwise unpleasant taste has prevented administration in typical lozenge or gum form. As indicated above, the length of time that the lozenge or gum may remain in the mouth and provide sustained release of a flavoring agent and/or beneficial agent, may be controlled in part by the appropriate selection of the water-insoluble polymer, essential oil, and/or water-soluble agent, and in part by the relative amounts of the polymer, essential oil, and/or water-soluble agent.

For instance, where it is desired to mask the taste of a beneficial agent, the polymer, essential oil, water-soluble agent, and beneficial agent may be admixed to form a slurry, a particulate (e.g., powder) material such as xylitol, sorbitol, or the like may be added to the slurry, and the slurry may further be admixed to form a coated granulated matrix. The coated granulated matrix may be compacted into a tablet or other dosage form as is and/or admixed with other excipients, described below, prior to preparation of a final dosage form.

### Sweeteners, Colorants, and Other Additives

In certain embodiments, the sustained release dosage form of the subject invention may also include one or more of a sweetener, a colorant and/or other additives. For instance, one or more sweeteners may be incorporated into the formulation so as to enhance the taste of the dosage form. Any sweetener well known in the art may be used. For example, the sweetener may be a sugar, e.g., sucrose, fructose, or dextrose, or may be a non-sugar sweetening agent, such as an agent that is formulated to both sweeten and to reduce the caloric intake as well as the likelihood of cavities or other dental related maladies.

Non-sugar sweetener agents that may be incorporated into a dosage form of the subject invention includes many well known artificial sweetening agents, such as, for instance, aspartame, saccharin, saccharin salts (e.g., sodium saccharin, calcium saccharin), sucralose, acesulfame-K (potassium acetosulfam), sorbitol, xylitol, stevioside, steviol, mannitol, erythritol, lactitol, alitame, miraculin, monellin, and thaumatin.

Where the dosage form is a lozenge, the sweetener may be incorporated or otherwise physically entrapped within the matrix produced by the admixing of the biocompatible and water-insoluble polymer with the essential oil component and/or water-soluble agent. Where the dosage form is a gum, the sweetener may be admixed with the dosage form matrix in such a manner so that although the sweetener is associated with the matrix of the dosage form, it is not entrapped therein. Thus, regardless of whether the dosage form is formulated as a lozenge or a gum, the ability of the dosage form to gradually release the essential oil and/or an included beneficial agent over a prolonged period of time is not substantially affected.

Additionally, the dosage form may also include a colorant and/or other conventional additives. Although some essential oils are already colored and, therefore, provide a given colored tint to the dosage form (e.g., peppermint oil imparts a yellow color and cinnamon oil imparts a brown color to the dosage form), in certain embodiments, this color may be changed and/or a new color may be added to the dosage form. For instance, without an added colorant, and in the absence of a colored flavoring agent, the lozenge and/or gum dosage form of the subject invention may be off-white or slightly darker, and may have some degree of translucence. Accordingly, a colorant may be added if a colored dosage form is desired.

Suitable colorants include natural colorants, e.g., pigments and dyes obtained from mineral, plant, and/or animal sources. Examples of natural colorants include red ferric oxide, yellow ferric oxide, annattenes, alizarin, indigo, rutin, and quercetin. Synthetic colorants may also be used and may include an FD&C or D&C dye, e.g., an approved dye selected from the so-called “coal-tar” dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a “lake” thereof, i.e., an aluminum or calcium salt thereof. Suitable colorants may be food colorants in the “GRAS” (Generally Regarded As Safe) category.

Another optional additive includes a release rate modifier, particularly release rate accelerants that also serve as softening agents, such as water-soluble polymers (e.g., MC, HPC, HPMC, etc.) and ingestible solvents (e.g., ethyl acetate, ethanol, glycerol, glycerol esters, etc.).

A further optional additive includes adhesion modifiers (including adhesion-increasing agents and adhesion-reducing agents), such as ingestible solvents (e.g., ethyl acetate and ethanol increase tack when admixed with ethylcellulose), mineral oil and vegetable oils (which tend to decrease tack when admixed with ethylcellulose), and additional polymers and polymer compositions, including polymers typically used to form hydrogels, e.g., ethylene vinyl acetate, polyvinyl alcohol, polyvinyl pyrrolidone, cellulose acetate, cellulose diacetate, and other cellulose esters, which may increase or decrease tack depending on the particular polymer or polymer composition.

Additional optional additives include: flavor stabilizers (e.g., starches), flavor diluents (e.g., ingestible solvents, as above), pH-adjusting agents (e.g., acids, bases, buffer systems), preservatives (e.g., antioxidants, antimicrobial agents, etc.), other binders to increase cohesiveness and promote more gradual dissolution of the dosage form (e.g., polycarbophil, polyethylene oxide, gum arabic, stearic acid), disintegrants for use in preparing quickly releasing and disintegrating dosage forms (e.g., glycerol, sugars, other polyols, etc.), lubricants, and fillers (e.g., maltodextrin, microcrystalline cellulose, lactose, mannitol, etc.). In certain embodiments, a dosage form of the subject invention does not include an absorbing agent, such as fume silica, or a release enhancer like polyethylene glycol, such as polyethylene glycol 300-6000, such as PEG 4000.

Enhancers may also be included so as to increase the permeation of a beneficial agent (if included) into the tissues of the oral cavity (e.g., in the administration of anti-inflammatory and/or antibiotic agents to treat oral mucositis, cold sores, periodontal disease, and pain following surgeries of the oral cavity or gums) and/or through the oral mucosa and into the bloodstream, to achieve enhanced systemic levels of a beneficial agent (as in sublingual drug administration) that has low oral bioavailability and does not readily penetrate through mucosal tissue. For instance, Methyl sulfonyl methane (“MSM”) may be included as an enhancer.

### Other Beneficial Agents, Conditions to be Treated, and Methods of Use

In certain embodiments, the sustained release dosage form of the subject invention includes one or more beneficial agents. Any suitable beneficial agent may be used to treat any adverse condition capable of being treated by the delivery of a beneficial agent to an oral cavity. In certain embodiments, a suitable beneficial agent may be one that is effective to produce a beneficial result. For instance, a beneficial agent may be one that when administered in the sustained release dosage form of the subject invention is effective for promoting at least a reduction in the severity and/or frequency of a symptom of an adverse condition. Accordingly, a beneficial agent may be one that is capable of masking and/or treating and/or preventing an adverse condition, which may or may not be a clinically symptomatic condition, in an individual by masking, inhibiting or causing the regression of the condition or its symptoms.

In certain embodiments, a beneficial agent may be any chemical compound, complex or composition that exhibits a desirable (e.g., beneficial) effect. Additionally, a beneficial agent may be a pharmaceutically acceptable derivative of a beneficial chemical compound, complex or composition, including, but not limited to, salts, esters, amides, prodrugs, active metabolites, isomers, analogs, and the like.

Accordingly, a beneficial agent that may be delivered using a dosage form of the subject invention may be selected from one or more of any of the following classes of agents including, but not limited to: analgesic agents, anesthetic agents (including local anesthetic agents for numbing a painful region within the mouth), anti-anginal agents, antiarthritic agents, anti-arrhythmic agents, antiasthmatic agents, anti-BPH agents, anticancer agents, anticholinergic agents, anticoagulants, anticonvulsants, antidepressants, antidiabetic agents, antidiarrheals, anti-epileptic agents, antifungal agents, antigout agents, antihelminthic agents, antihistamines, antihypertensive agents, antiinflammatory agents, antimalarial agents, antimicrobial agents (including local antibiotics for treatment of an infection of the gum or elsewhere within the oral cavity), antimigraine agents, antimuscarinic agents, antinauseants, antineoplastic agents, antiosteoporosis agents, antiparkinsonism agents, antiprotozoal agents, antipruritics, antipsychotic agents, antipyretics, antispasmodics, antithyroid agents, antitubercular agents, antiulcer agents, anti-urinary incontinence agents, antiviral agents, anxiolytics, attention deficit disorder (ADD) and attention deficit hyperactivity disorder (ADHD) drugs, calcium channel blockers, cardiac inotropic agents, beta-blockers, central nervous system stimulants, cognition enhancers, corticosteroids, COX-2 inhibitors, cough and cold preparations, diet aids, diuretics, gastrointestinal agents, genetic materials, histamine receptor antagonists, hormonolytics, hypnotics, hypoglycemic agents, immunosuppressants, keratolytics, leukotriene inhibitors, lipid-regulating agents, macrolides, mitotic inhibitors, muscle relaxants, narcotic antagonists, neuroleptic agents, nicotine, nutritional agents, such as vitamins, essential amino acids, and fatty acids; parasympatholytic agents, sedatives, sex hormones, sympathomimetic agents, tranquilizers, vasodilators, vitamins, other associated agents (e.g., polymers) that produce a desired effect in the mouth, and combinations thereof. However, in certain embodiments a beneficial agent may be any agent or any chemical compound, complex or composition that exhibits a desirable (e.g., beneficial) effect, with the proviso that the beneficial agent is not a herbal medication. By “herbal medication” is meant any medication that is derived from botanical materials or a biologically active extract of these materials.

As will be readily understood by those of skill in the art, any of the aforementioned beneficial agents may be administered alone or in combination with one another. Beneficial agents administered in combination may be from the same therapeutic class (e.g., two different diet aids) or from different therapeutic classes (e.g., a decongestant and a vitamin).

The beneficial agent may be administered to provide a local, topical effect, within the oral cavity (e.g., as a topical anti-infective or anesthetic), or to achieve a systemic effect by passing through the mucosal membranes within the oral cavity and into an individual's blood stream. The appropriate amount of any beneficial agent in the dosage form will depend on the particular agent and the intended daily dose, and presumes that one to twelve, or two to ten, including four to eight, such as five to six, dosage forms will be consumed on a daily basis. Unless explicitly indicated herein, it is to be understood that appropriate daily doses for the various agents will be known to those of ordinary skill in the art of pharmaceutical formulation and pharmacology and/or can be found in the pertinent texts and literature.

The dosage forms of the subject invention, in certain embodiments, are well-suited to administer beneficial agents the efficacy of which increases as a result of an extended residence time in the oral cavity, thereby resulting in greater oral mucosal absorption of any particular agent. Such agents include, by way of example: glutathione and other agents that are degraded in or otherwise rendered unstable in the gastrointestinal tract; coenzyme Q10 and xylitol, for instance, in the treatment of periodontal disease and/or adverse systemic conditions; aspirin and nonsteroidal anti-inflammatory agents; antinauseants, anti-emetic agents, opioid analgesics, and other medications which the stomach may not tolerate, and allergy medications, such as for the rapid relief of allergic symptoms (e.g., diphenhydramine).

The dosage forms of the subject invention are also useful in adult and pediatric applications, e.g., in the administration of cough and cold medications to adults or children. In this way, the need for medicated tablets, which some adults and children often find difficult to swallow, is avoided.

Other beneficial agents that may be included are agents for combating xerostomia, dry mouth and/or halitosis, as well as cold remedy agents, local anesthetics, local anti-infective agents, diet aids, fluoride-releasing compounds and other agents exhibiting utility in the dental context, and nicotine. For instance, xerostomia is a condition that results in dry mouth. The dry mouth may result from a lack of saliva. A subject suffering from xerostomia may have a physical condition whereby the salivary glands, ducts, and/or nerves connected therewith are in some way deficient such that saliva is not produced. However, xerostomia and/or dry mouth may also result as a symptom of other underlying diseases, such as Sjourgren's syndrome, Eaton-Lambert syndrome, diabetes and/or may result as a side effect from taking other drugs, medications, or result from anxiety, nervousness, and/or dehydration. In all of these situations, a dosage form of the present invention is useful in treating the symptoms of the dry mouth that results from one or more of these underlying conditions.

Specifically, a dosage form of the subject invention may be delivered by itself or in conjunction with a liquid (e.g., water) to produce a lubricated environment within an oral cavity (e.g., by coating one or more mucosal surfaces of the mouth) and thereby relieve dry mouth. Additionally, a dosage form of the subject invention may also be formulated so as to include a beneficial agent, such as xanthan gum, polycarbophil, polyethylene oxide, hydroxypropylmethylcellulose (HPMC), pectin, guar gum, and the like, that may further treat or prevent an underlying condition such as xerostomia, Sjogren's syndrome, Eaton-Lambert syndrome, diabetes, and the like. For instance, a suitable saliva substitute, such as methyl cellulose, carboxymethyl cellulose, xylitol, pilocarpine, and the like, may be added as a beneficial agent to a dosage form of the subject invention to both produce a lubricated condition in the mouth as well as to treat or reduce the underlying symptoms of xerostomia when delivered to the mouth.

Additionally, other active agents may also be included such as beneficial agents for the treatment of the common cold. For example, a suitable beneficial agent that may be included as a cold remedy includes, but are not limited to: sources of Zn²⁺, i.e., ionizable zinc compounds; vitamins, including vitamin C optionally combined with one or more B vitamins; and herbal extracts, such as echinacea and golden seal. For instance, ionizable zinc compounds when formulated and delivered in accordance with the subject invention are useful for reducing the duration and/or symptoms of common colds, managing upper respiratory allergy, as nutritional agents, and in treating halitosis, e.g., for masking, reducing or eliminating bad breath.

Accordingly, a suitable ionizable zinc compound may be an inorganic or organic complex; examples of suitable complexes include zinc gluconate, acetate, chloride, propionate, butyrate, n-butyrate, beta-hydroxybutyrate, benzoate, formate, and sulfate, although zinc acetate and gluconate may be used for reasons of stability, acidity in an aqueous environment (and thus potential toxicity), and suitability for sustained release in the present formulations. In this regard, the prolonged release characteristics of the zinc containing lozenges and/or gums of the subject invention are superior to those known in the prior art.

Specifically, conventional zinc lozenges last only minutes before being absorbed and thus they have a limited time frame in which to exert a maximal therapeutic effect. The beneficial agent containing lozenges and gums of the subject invention, however, are formulated so as to maximize the time period during which the zinc compound is released as well as to minimize the unpleasant, bitter taste of many zinc-containing compounds. Additionally, for the treatment of colds, combinations of ionizable zinc compounds with other cold remedies, e.g., vitamin C, herbal remedies, decongestants, etc., are also useful.

Generally, the amount of ionic zinc (i.e., Zn²⁺) in a dosage form of the invention is in the range of about 1 mg to about 50 mg, typically in the range of about 5 mg to about 40 mg, such as in the range of about 15 mg to about 35 mg (these ranges correspond to about 12.8 mg to about 640 mg, typically about 64 mg to about 512, for instance, about 192 mg to about 448 mg zinc gluconate, insofar as ionic zinc represents approximately 12.8 wt. % of zinc gluconate).

In the treatment of halitosis, the dosage forms need not include a beneficial agent, insofar as the essential oil component may act as a flavoring agent itself and may mask or otherwise reduce bad breath for extended time periods. However, incorporation of an additional beneficial agent such as an ionizable zinc compound may also serve to combat halitosis. Specifically, while the essential oil component or flavoring agent masks the odor associated with halitosis, a zinc compound, as discussed above, such as zinc acetate or zinc gluconate, and may act by combining with any volatile sulfur compounds that may function to produce halitosis.

Other agents for reducing or eliminating halitosis can also be incorporated into the dosage form, and may or may not target a particular cause of the problem (e.g., infections of the mouth, nasal or sinus conditions, gastrointestinal disorders, diabetes, etc.). For example, anti-infective agents, such as triclosan or phenol, may be included. In contrast to breath mints and other breath fresheners known in the art, the present dosage forms, containing a flavoring agent and optionally one or more additional beneficial agents for treating halitosis, can reduce bad breath for up to several hours or more. Additionally, if non-sugar sweeteners are included the dosage form may retain a pleasant, sweet taste for an extended time period, and yet will not promote dental caries.

Further, the dosage forms may include a local anesthetic agent, for instance, to reduce sore throat pain, and/or a local anti-infective agent, for instance, to eliminate any bacteria or virii, such as, bacteria associated with a sore throat. Local anesthetics include, for example, menthol, benzocaine, bupivacaine, butambenpicrate, chlorprocaine, cocaine, dibucaine, dimethisoquin, dyclonine, etidocaine, hexylcaine, hexylresorcinol, ketarine, lidocaine, mepivacaine, phenol, phenolate, pramoxine, procaine, ropavacaine, tetracaine, tripelennamine, xylocalne, and pharmaceutically acceptable salts thereof (e.g., dimethisoquin hydrochloride, pramoxine hydrochloride) while representative anti-infective agents include amylmetacresol, benzalkonium, cetylpyridinium, chlorhexidine, dequilinium, domiphen, dichlorobenzyl alcohol, phenol, and tyrothicin. Of course, a source of zinc ion, such as zinc acetate or zinc gluconate, may also be incorporated into a lozenge or gum of the invention for reducing sore throat pain, insofar as such compounds exhibit therapeutic activity. It will be appreciated that these dosage forms are also useful in treating and/or reducing pain associated with local viruses of the mouth, which may often be manifested as sores or lesions (e.g., those associated with herpes infection), or with various disorders of the tongue.

Accordingly, the dosage forms of the invention may also be useful in treating oral sores, including cold sores and oral mucositis. Use of anti-inflammatory agents and antibiotics to treat or prevent cold sores and oral mucositis has, in the past, proven difficult because ointments and mouth washes result in limited contact of the agent with the affected tissue. By contrast, the dosage forms of the subject invention may provide extended contact of the beneficial agent (e.g., dexamethasone) with the affected tissue, and thereby reduce the length of time required for a sore to heal. In the treatment of oral sores, a local anesthetic agent as those enumerated above may also be advantageously incorporated into a dosage form of the subject invention.

A diet aid may additionally be included as a beneficial agent of a dosage form of the subject invention. It is to be noted that even without the addition of a diet aid, the dosage forms of the subject invention may facilitate weight reduction. For instance, where a food flavor or citrus type essential oil is included in a sustained release formulation of the subject invention, the flavor may mimic the taste of food in the mouth. Incorporation of a diet aid, however, may further enhance weight reduction. A diet aid may include any agent that assists an individual to reduce the intake of food, regardless of mechanism. Therefore, diet aids for use herein may suppress appetite, give the feeling of “fullness,” and/or increase metabolism. While any diet aid may be administered to an individual using the present dosage forms, exemplary diet aids include 5-hydroxytryptophan, tyrosine, phenylalanine, pseudoephedrine, ephedrine, phenylpropanolamine, chromium picolinate, aspirin, benzocaine, carnitine, and caffeine. Certain herbal preparations, mixtures, and extracts are also suitable diet aids, and include, without limitation, guarana and ma huang.

Additionally, the beneficial agent may be one that promotes healthy teeth and gums, or that exhibits other utility in the “dental” context. For instance, a fluoride-releasing dosage form may be prepared by incorporating a source of fluoride ion as a beneficial agent. Fluoride-releasing agents are well known and include sodium monofluorophosphate, sodium fluoride, and stannous fluoride. Fluoride-containing dosage forms may contain xylitol as a sweetener, as xylitol may potentiate the action of the fluoride. Also, a local anesthetic agent, as described above, can provide for desensitization within the mouth, to alleviate a toothache or other pain associated with a condition or disorder of the gums, or the pain or discomfort that may follow a dental procedure.

Another beneficial agent that may be included is nicotine, which may be in the form of the free base or an acid addition salt thereof. As an aid to smoking cessation, nicotine has been incorporated into gums and other drug delivery systems in the form of the acid addition salt, in large part to offset the bitter and unpleasant taste of the free base. Because the essential oil component of the subject invention may include a flavor component, the present dosage forms provide for very effective taste-masking with respect to a wide variety of beneficial agents, such as nicotine, which can be incorporated into a dosage form of the subject invention and released as the free base (or the salt) over a prolonged period of time.

Since the base is more readily delivered across the mucosal membrane than the salt form of the drug, the invention enables delivery of a lower dose of nicotine, particularly when the dosage form is a lozenge. Suitable gums and lozenges may contain 2 mg, 4 mg, or 10 mg nicotine. That is, a lozenge of the invention may contain less than about 5 mg of nicotine, for instance, 0.1 to 2 mg, including 0.25 to 1.5 mg, while nevertheless providing the desired therapeutic effect. With nicotine-containing dosage forms, it may be desirable to incorporate or disperse the nicotine in an excipient that reduces the volatility of the drug (e.g., mannitol, microcrystalline cellulose, colloidal silica), unless the nicotine is in the form of an acid addition salt. A sweetener may also be included to provide taste-masking. While any of the above-mentioned sweeteners may be used, a suitable sweetener in nicotine lozenges is sucralose.

While the above discussion refers to certain dosage forms of the invention as “lozenges,” it is to be understood that the term encompasses lozenge-type dosage forms that may or may not have some degree of adhesion. A suitable dosage form of the subject invention in this regard may be substantially flat and adhere to the gum or teeth to both produce a lubricated condition within an oral cavity and to deliver a beneficial agent, e.g., an anti-infective agent including any of the local anti-infective agents set forth above, a local anesthetic agent, including those exemplified previously, or an anti-inflammatory agent.

Anti-inflammatory agents that may be included as a beneficial agents in a dosage form of the subject invention include by way of example: NSAIDS (nonsteroidal anti-inflammatory agents), such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen and tiaprofenic acid; acetylsalicylic acid, apazone, diclofenac, difenpiramide, diflunisal, etodolac, flufenamic acid, indomethacin, ketorolac, meclofenamate, mefenamic acid, nabumetone, phenylbutazone, piroxicam, sulindac, and tolmetin, and corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, methylprednisolone, clobetasol, betamethasone fluocinonide, mometasone, triamcinolone acetonide, and the like.

Any of the beneficial agents may be in the form of a salt, ester, amide, prodrug, active metabolite, isomer, analog, or the like, provided that the salt, ester, amide, prodrug, active metabolite, isomer, or analog is pharmaceutically acceptable and retains at least some degree of the desired activity. Salts, esters, amides, prodrugs, metabolites, analogs, and other derivatives of the beneficial agents herein may be prepared using standard procedures known to those skilled in the art of synthetic organic chemistry and described, for example, by J. March, Advanced Organic Chemistry: Reactions, Mechanisms and Structure, 4th Edition (New York: Wiley-Interscience, 1992).

For example, acid addition salts are prepared from a beneficial agent in the form of a free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties that may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the beneficial agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

Accordingly, in one aspect, the subject invention is directed to a method for producing a desired effect or condition within an oral cavity (e.g., the mouth) of a subject by administering a dosage form of the subject invention to the mouth. For instance, in certain embodiments, the subject invention provides a method for delivering an essential oil component within an oral cavity of a user. In other embodiments, the subject invention provides a method for masking, treating, preventing, or otherwise ameliorating an adverse condition, such as those described above, in a subject by administering a dosage form of the subject invention to the mouth wherein the dosage form includes a beneficial agent, such as those described above. In certain embodiments, the subject invention is directed to a method of both delivering an essential oil component within the mouth and/or masking, treating, preventing, or otherwise ameliorating an adverse condition therein, by administering a dosage form of the subject invention to the mouth.

Hence, in certain embodiments, a method is provided for using the presently disclosed dosage forms in the administration of beneficial agents to the oral cavity, e.g., mouth of an individual, such as a human individual. Administration may be local, such that the beneficial agent exhibits its desired effect within the oral cavity. Administration may also be systemic, in which case delivery of the beneficial agent is transmucosal, i.e., the beneficial agent passes through the mucosal lining of the oral cavity and into the bloodstream, such that the beneficial agent then exhibits its desired effect systemically. In one embodiment, the method provides for sustained release of a flavoring agent in the mouth, e.g., in the treatment of halitosis.

In certain embodiments, a method for treating the common cold is provided. In certain embodiments, a method for treating a sore throat is provided. In certain embodiments, a method for facilitating weight loss is provided. In certain embodiments, a method for assisting an individual in quitting smoking is provided. In certain embodiments, a method for delivering a beneficial agent to a mucosal surface within the mouth is provided.

Accordingly, in certain embodiments, the methods of the subject invention include, administering to an individual in need of treatment a dosage form that includes an admixture of ethylcellulose, such as an ethylcellulose having a solution viscosity in the range of approximately 90 to 110 cP as determined at 25° C. using a 5 wt. % aqueous solution; an essential oil component, selected from essential oils, individual terpenes, and individual sesquiterpenes, wherein the weight ratio of the ethylcellulose to the flavoring agent is in the range of approximately 1:1.5 to 1.5:1; and a beneficial agent, for instance, an ionizable zinc compound, a local anesthetic agent, a diet aid, nicotine, or other beneficial agent set forth herein. As noted above, a sweetening agent may also be included.

For instance, in certain embodiments, a method for delivering an essential oil component to an oral cavity (e.g., the mouth) of a subject and/or masking, treating, preventing, or otherwise ameliorating an adverse condition therein is provided, wherein a dosage form of the subject invention, including a water-insoluble polymer, an essential oil component, and/or an effective amount of a beneficial agent is administered to the mouth of the user. The dosage form may be in the form of a lozenge or gum, wherein the effective amount of the essential oil component and/or beneficial agent, as well as the type of the essential oil and/or water-soluble component, are selected so as to provide the lozenge or gum with the capability of providing a pleasant taste and/or smell within the mouth, and, if included, the amount of the beneficial agent is selected so as to effectively mask, treat, prevent, or otherwise ameliorate an adverse condition, when the lozenge or gum is positioned therein.

Accordingly, where desired, e.g., to ameliorate an undesired condition, an effective amount of a beneficial agent may be included in the dosage form to be delivered to the oral cavity. For instance, a beneficial amount of a beneficial agent, such as an agent for masking and/or treating xerostomia, dry mouth, halitosis, a common cold, a local antibiotic, a local anesthetic agent, pilocarpine, vitamin C, a source of Zn2+, zinc gluconate, zinc acetate, chloride, propionate, butyrate, n-butyrate, β-hydroxybutyrate, benzoate, formate, sulfate, a diet aid, 5-hydroxytryptophan, tyrosine, phenylalanine, pseudoephedrine, ephedrine, phenylpropanolamine, chromium picolinate, aspirin, caffeine, nicotine, a herbal mixture or extract thereof, guarana and ma huang, a source of fluoride ion, and combinations thereof, may be included in the dosage form. However, although the dosage form may include a beneficial agent, in certain embodiments, the beneficial agent is not a herbal medication, herbal mixture, or extracts of such materials. In this manner, in certain embodiments, a method for masking, treating, preventing and/or ameliorating the symptoms of such conditions of dry mouth, xerostomia, halitosis, a cold, an infection, a sore throat, obesity, an addiction to smoking, cavities and/or the like is provided.

Specifically, a dosage form for use in accordance with above described methods may include a water-insoluble polymer that has an average particle size diameter in the range of about 1 micron to about 250 microns and/or the viscosity of the polymer may, in some instances, be in the range of about 90 cP to about 110 cP. For instance, in certain embodiments, the water-insoluble polymer may include ethylcellulose.

Additionally, in certain embodiments, the essential oil component of the lozenge or gum may include an essential oil such as: a citrus oil, lemon oil, lime oil, neroli oil, orange oil, a mint oil, peppermint oil, spearmint oil, anise oil, cardamom oil, cinnamon oil, clove oil, coriander oil, eucalyptus oil, fennel oil, lemongrass oil, nutmeg oil, eriodictyon fluid extract, glycyrrhiza extract, or combinations thereof. In certain embodiments, the weight ratio of the biocompatible, water-insoluble polymer to essential oil component of the dosage form may be in the range of about 1:5 to 2:1.

Further, in certain embodiments, a water-soluble component may be included in the lozenge or gum of the dosage form and may include: gum arabic, or the like, in a sufficient amount such that the combination of the ethylcellulose, essential oil component, and water-soluble component form a matrix composition that, when positioned in the mouth of a subject, the matrix composition slowly dissolves gradually releasing the essential oil and/or a beneficial agent in to the subject's mouth and thereby producing the desired effect, such as producing a pleasant taste or smell within the mouth or ameliorating an adverse condition therein. Accordingly, in certain embodiments, a dosage form of the subject invention may include a beneficial agent as described above. In certain embodiments, a dosage form of the subject invention may include a beneficial agent, as described above, with the proviso that the beneficial agent is not a herbal medication, such as a medication derived from botanical materials and/or a biologically active extract of such materials. However, in certain embodiments, the dosage form includes a water-insoluble polymer, an essential oil component, and may include a water soluble component or other additives but does not include a beneficial agent. Specifically, in certain embodiments, a dosage form of the subject invention does not include a herbal medication, such as a medication derived from botanical materials and/or a biologically active extract of such materials.

### Methods of Manufacture

In certain embodiments, a dosage form of the subject invention may be prepared by admixing together a biocompatible polymer, an essential oil component, and/or a water-soluble component, and/or a beneficial agent, and/or any additional components, including sweeteners, colorants, other additives discussed herein. Admixture can generally be carried out at room temperature and ambient humidity, unless a particular beneficial agent or other component of the dosage form (e.g., lozenge) requires a protected environment, a lower temperature, or lower humidity. Using the appropriate weight ratio of the polymer to the essential oil and water-soluble component (if included), as discussed supra, admixture of the components results in a pliable dosage form that can be formed into a roll or sheet.

After allowing the composition to set, typically over a 24-hour period, the lozenges are then created by cutting of the roll or die cutting of the sheet. In a preferred embodiment, the mixture of the components is compressed to form lozenges. For example, the mixture can be compressed in a two-part lozenge-shaped mold, wherein after the mixture is added to a recess within the lower half of the mold, the upper half is aligned therewith and pressure is applied to compress the mixture. In certain embodiments the pressure applied is more than 10 Torr, such as 15 Torr or above, such as 25 Torr and above, including about 50 Torr or about 100 Torr to about 500 Torr or more. However, in certain embodiments, the pressure applied is less than 10 Torr, such as about 9 Torr or less, such as about 8 Torr or less, for instance, 5 Torr or less, including 3 Torr or less. Compressed lozenges can be made so as to remain intact within the mouth for extended time periods, on the order of five hours or more. It will be appreciated, however, that the present process can be tailored to provide compressed lozenges that degrade more quickly, for example by varying the proportion of flavoring agent(s) and/or excipients.

If a somewhat tacky lozenge is desired, e.g., a dosage form that adheres to the buccal mucosa for delivery of a beneficial agent, the same procedures are followed except that a lower molecular weight water-insoluble polymer is used to impart adhesive strength to the lozenge by virtue of the tacky surface provided. Alternatively, or in addition, one or more adhesive polymers can be incorporated into the lozenge formulation to provide the desired degree of adhesion, as described elsewhere herein.

Chewing gums may be prepared by first formulating the wet matrix as described above, i.e., by admixing the water-insoluble polymer and the flavoring agent. Then, the matrix, along with any additional components, e.g., sweeteners, colorants, or other additives, is admixed with a selected chewing gum base as described earlier herein. Mixing may be effected using any suitable mixing device, e.g., a ribbon blender. The resultant chewing gum is then manufactured into strips or tablets of a desired size.

The dosage forms so prepared are individually packaged in a manner that promotes shelf life and maximizes the stability of the flavoring agent. These requirements translate into a package design in which both the air space and exposed surface area of the lozenge are minimized, and in which the packaging material used has very low permeability to vapor. A plastic-lined foil, wherein the plastic is a low permeability material, is optimal. Ideally, the packaging material should be in contact with at least 85% of the surface of the lozenge to minimize loss of flavor, and packaging materials that do not transmit organic vapors are optimal. For example, polyolefinic materials such as poly(vinylidene chloride), polyethylene (including low density and higher density polyethylenes), polypropylene, and copolymers thereof represent suitable packaging materials.

The dosage forms of the invention may be prepared in any number of shapes and sizes, and the invention is not limited in this regard. Different shapes and sizes may be desirable for different applications. Typical dimensions, however, are on the order of 0.4″×0.5″×0.2″ for lozenges, while lozenge weight is generally in the range of about 0.4 to 0.8 g. For chewing gums, the dimensions will generally be somewhat different, insofar as flat, elongated strips and/or larger tablets are often preferred. It is to be understood that while the invention has been described in conjunction with specific embodiments thereof, the description above as well as the examples that follow are intended to illustrate and not limit the scope of the invention. Other aspects, advantages and modifications within the scope of the invention will be apparent to those skilled in the art to which the invention pertains.

### EXAMPLES

The following examples are put forth so as to provide those skilled in the art with a complete disclosure and description of how to make and use embodiments in accordance with the invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example I

### Preparation of Flavored Lozenges

Lozenges were prepared by mixing 0.124 g (25.8%) ethylcellulose, such as ETHOCEL® Standard 100 Premium; 0.0058 g (1.2%) wintergreen and 0.102 g (21.2%) peppermint oil; 0.093 g (19.3%) gum arabic; 0.032 g (6.6%) sucralose, and 0.032 g (6.6%) xylitol together along with other additives (see Table I, below) at room temperature and ambient humidity. Admixture of the components resulted in a soft, wet composition that was formed into a lozenge via a press and allowed to set for 24 hours. Then, lozenges each weighing 0.2 g were cut.

In the oral environment of a human test subject, after 2 hours in the mouth, the lozenges entirely dissolved thereby releasing the essential oil component and the other components into the aqueous environment of the oral cavity.

The constituents of the dosage form included:

TABLE I

| | Grams | % |
|---|---|---|
| Ethylcellulose | 0.124 | 25.8 |
| Sucralose | 0.032 | 6.6 |
| Menthol | 0.047 | 9.8 |
| Gum Arabic | 0.093 | 19.3 |
| Sodium bicarbonate | 0.013 | 2.7 |
| Eucalyptol | 0.0048 | 1.0 |
| Thymol | 0.0048 | 1.0 |
| Wintergreen | 0.0058 | 1.2 |
| Glycerol | 0.013 | 2.7 |
| Peppermint oil | 0.102 | 21.2 |
| xylitol | 0.032 | 6.6 |
| Other additives | 0.01 | 2.1 |
| Total lozenge | 0.4814 | 100.0 |
| weight | | |

### Example II

### Preparation of Zinc Gluconate Lozenges

Lozenges were prepared by mixing 0.124 g (25.8%) ethylcellulose, such as ETHOCEL® Standard 100 Premium; 0.0058 g (1.2%) wintergreen and 0.102 g (21.2%) peppermint oil; 0.093 g (19.3%) gum arabic; 0.01 g (2.1%) zinc gluconate; 0.032 g (6.6%) sucralose; and 0.032 g (6.6%) xylitol together along with other additives (see Table II, below) at room temperature and ambient humidity. Admixture of the components resulted in a soft, wet composition that was formed into a lozenge via a press and allowed to set for 24 hours. Then, lozenges each weighing 0.2 g were cut.

In the oral environment of a human test subject, after 2 hours in the mouth, the lozenges entirely dissolved thereby releasing the essential oil component, zinc, and the other components into the aqueous environment of the oral cavity.

The constituents of the dosage form included:

TABLE II

| | Grams | % |
|---|---|---|
| Ethylcellulose | 0.124 | 25.8 |
| Sucralose | 0.032 | 6.7 |
| Wintergreen | 0.0024 | 0.5 |
| Sodium bicarbonate | 0.013 | 2.7 |
| Eucalyptol | 0.0015 | 0.3 |
| Thymol | 0.0015 | 0.3 |
| Glycerol | 0.013 | 2.7 |
| Zinc Gluconate | 0.01 | 2.1 |
| Gum arabic | 0.124 | 25.8 |
| Peppermint oil | 0.1275 | 26.5 |
| xylitol | 0.032 | 6.7 |
| Total lozenge weight | 0.4809 | 100 |

### Example III

### Additional Preparation of Zinc Gluconate Lozenges

Lozenges were prepared by mixing 0.124 g (25.8%) ethylcellulose, such as ETHOCEL® Standard 100 Premium; 0.0058 g (1.2%) wintergreen and 0.102 g (21.2%) peppermint oil; 0.093 g (19.3%) gum arabic; 0.01 g (2.1%) zinc gluconate; 0.032 g (6.7%) sucralose; and 0.032 g (6.7%) xylitol together along with other additives (see Table III, below) at room temperature and ambient humidity. Admixture of the components resulted in a soft, wet composition that was formed into a lozenge via a press and allowed to set for 24 hours. Then, lozenges each weighing 0.2 g were cut.

In the oral environment of a human test subject, after 2 hours in the mouth, the lozenges entirely dissolved thereby releasing the essential oil component, zinc, and the other components into the aqueous environment of the oral cavity.

The constituents of the dosage form included:

TABLE III

| | Grams | % |
|---|---|---|
| Ethylcellulose | 0.124 | 25.8 |
| Sucralose | 0.032 | 6.7 |
| Wintergreen | 0.0058 | 1.2 |
| Sodium bicarbonate | 0.013 | 2.7 |
| Eucalyptol | 0.0048 | 1.0 |
| Thymol | 0.0048 | 1.0 |
| Glycerol | 0.013 | 2.7 |
| Zinc Gluconate | 0.01 | 2.1 |
| Gum arabic | 0.093 | 19.3 |
| Peppermint oil | 0.102 | 21.2 |
| Xylitol | 0.032 | 6.7 |
| Menthol | 0.047 | 9.8 |
| Total lozenge weight | 0.4814 | 100 |

### Example IV

### Effectiveness of the Subject Lozenges of the Invention

12 subjects received a dental prophylaxis and were instructed to brush only the lingual surface of their teeth (e.g., inside, in contact with the tongue) for up to 60 seconds, twice a day, using a standard sodium fluoride dentifrice and an American Dental Association (ADA) manual reference toothbrush. Subjects were instructed to abstain from flossing, using chewing gum, toothpicks, fresh breath sprays, or performing any other oral hygiene after the baseline visit to the end of the study.

Week 1: At Day 1 (Monday) subjects received a polishing of the lingual and buccal surfaces of their teeth followed by a Plaque Index Exam to assure that all teeth surfaces were free of plaque. Then they were assigned randomly to one of the 2 study articles A or B and given instructions on how to use them.

Study Article A: If the subject was assigned to test article A (DENTIVA™), they were instructed to use one DENTIVA™ 3 times a day, after breakfast, lunch and dinner and keep the lozenge in the mouth for at least one hour without biting on or chewing it. The DENTIVA™ formulation used was identical to that set forth in Table II, above.

Study Article B: If the subject was assigned to test article B (LISTERINE®), they were instructed to use 20 ml of the mouth rinse product for 30 seconds in the morning and evening after the prescribed tooth brushing procedure.

Thursday—Day 4: In the morning of Day 4 (Thursday) subjects brushed their teeth as instructed only on the lingual side, and follow the instructions (have a lozenge or gargle with LISTERINE®) before they returned to the dental office. Here they received a disclosing fluid and were examined for plaque build up using the modified Turesky scoring system.

Week 2: The same procedure was repeated as in the first week, only this time they received the other Test Article.

Results: The maxillary and mandibular PI scores were averaged per patients and test article and are shown in Table IV below. On average each patient had almost 1 point less bio-film build up when on DENTIVA™ then when on LISTERINE®. See Table III, below.

TABLE IV

| | | Subject #: | | | | | | | | | | | | | |
|---|---|---|---|---|---|---|---|---|---|---|---|---|---|---|---|
| | | 1 | 2 | 3 | 4 | 5 | 6 | 7 | 8 | 9 | 10 | 11 | 12 | AVG | STD |
| Maxillary | F - Dentiva | 1.7 | 1.9 | 1.9 | 1.9 | 3.1 | 2.3 | 2.4 | 2.2 | 2.5 | 2.1 | 1.9 | 1.3 | 2.1 | 0.5 |
| | F - Listerine | 2.3 | 2.6 | 2.6 | 2.9 | 3.2 | 3.2 | 3.1 | 3.8 | 3.5 | 3.4 | 2.6 | 2.8 | 3.0 | 0.4 |
| Mandibular | F - Dentiva | 1.6 | 1.1 | 1.7 | 1.9 | 2.4 | 2.9 | 2.7 | 2.3 | 2.7 | 2.8 | 2.1 | 1.1 | 2.1 | 0.6 |
| | F - Listerine | 2.3 | 1.9 | 3.0 | 2.2 | 3.0 | 3.4 | 3.0 | 3.5 | 3.1 | 3.4 | 2.7 | 2.3 | 2.8 | 0.5 |

### Example V

### Effectiveness of the Subject Lozenges of the Invention

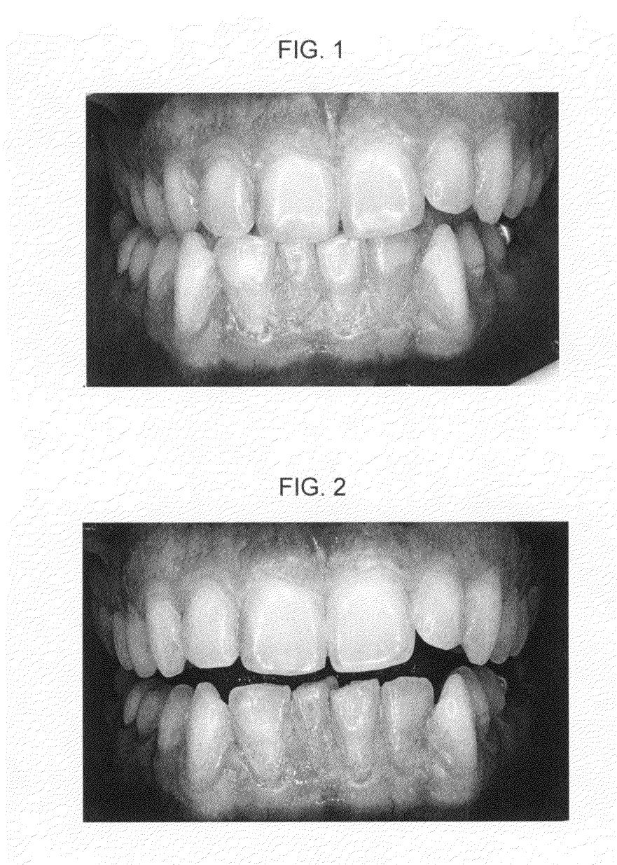

Using a protocol similar to that set forth above, a subject had his teeth cleaned and polished and then didn't brush facial tooth surfaces for 3 days (only lingual sides were brushed twice a day). During that time the subject gargled with LISTERINE® after each brushing. On Day 4 pictures were taken. See FIG. 1. Then the study was repeated one week later, this time the subject used DENTIVA™ after breakfast, lunch and dinner. The DENTIVA™ formulation used was identical to that set forth in Table II, above. Again pictures were taken on Day 4. See FIG. 2. As can be seen with reference to FIGS. 1 and 2 the subject had easily discernable brighter and thicker biofilms when on LISTERINE®, whereas biofilms were much thinner and covered less area of the teeth when DENTIVA™ was used. Additionally, the subject reported that overall mouth feel was much more agreeable with DENTIVA™.

### Example VI

### Oral Health Lozenges

To improve oral health by reducing bacteria, reducing biofilm, to adjust pH to neutral to prevent demineralization, and decrease odor in the mouth by capturing sulfhydryl compounds, the following formulation was prepared by mixing 0.62 g (25.8%) ethylcellulose, such as ETHOCEL® Standard 100 Premium; 0.0029 g (1.2%) wintergreen and 0.51 g (21.2%) peppermint oil; 0.465 g (19.3%) gum arabic; 0.05 g (2.1%) zinc gluconate; 0.16 g (6.6%) sucralose; and 0.16 g (6.6%) xylitol together along with other additives (see Table V, below) at room temperature and ambient humidity.

TABLE V

| | Grams | % |
|---|---|---|
| Ethylcellulose | 0.62 | 25.8 |
| Sucralose | 0.16 | 6.6 |
| Menthol | 0.235 | 9.8 |
| Gum Arabic | 0.465 | 19.3 |
| Sodium bicarbonate | 0.065 | 2.7 |
| Eucalyptol | 0.024 | 1.0 |
| Thymol | 0.024 | 1.0 |
| Wintergreen | 0.029 | 1.2 |
| Glycerol | 0.065 | 2.7 |
| Zinc Gluconate | 0.05 | 2.1 |
| Peppermint oil | 0.51 | 21.2 |
| xylitol | 0.16 | 6.6 |
| Total lozenge weight | 2.407 | 100.0 |

Accordingly, the lozenges were prepared according to the above by mixing the above components at room temperature and ambient humidity. The mixture of the components resulted in a soft, wet composition that was pressed into lozenge forms of about 0.42 g each. In the oral environment with multiple human test subjects, the lozenges dissolved by dissolution in about 1 hour, the exact time depending on the extent of movement of the lozenge in the mouth of the various subjects.

Such long lasting lozenge may be useful in that the added length of time the lozenge is in the mouth results in greater reduction of biofilm and bacteria. Typical mouthwash with essential oils or candy or gums with essential oils may have low residence times with sufficient essential oils which significantly decreases their ability to reduce biofilm and bacteria. The above lozenges of the subject invention overcame this by its levels of ingredients in a long lasting lozenge (formulated herein for about an hour to two hour dissolution rate).

### Example VII

### Fluoride Containing Lozenges

Many water supplies do not carry fluoride. Fluoride is well known to strengthen the teeth and thereby prevent caries. Accordingly, a fluoride lozenge may be useful in preventing caries in school age children as well as in the overall population, with added emphasis to the older population in risk of caries or for those with xerostomia. The following formulation was prepared, with ingredients that would, in addition to preventing caries, e.g., due in part to fluoride, would reduce biofilm, inhibit bacterial growth, adjust pH to prevent demineralization, and reduce bad breath. The following formulation was prepared by mixing 0.62 g (25.7%) ethylcellulose, such as ETHOCEL® Standard 100 Premium; 0.0029 g (1.2%) wintergreen and 0.51 g (21.2%) peppermint oil; 0.465 g (19.3%) gum arabic; 0.05 g (2.1%) zinc gluconate; 0.16 g (6.6%) sucralose; 0.16 g (6.6%) xylitol; and 0.001 g (0.04%) stannous fluoride together along with other additives (see Table VI, below) at room temperature and ambient humidity.

TABLE VI

| | Grams | % |
|---|---|---|
| Ethylcellulose (e.g. | 0.62 | 25.7 |
| ethocel 100) | | |
| Sucralose | 0.16 | 6.6 |
| Menthol | 0.235 | 9.8 |
| Gum Arabic | 0.465 | 19.3 |
| Sodium bicarbonate | 0.065 | 2.7 |
| Eucalyptol | 0.024 | 1.0 |
| Thymol | 0.024 | 1.0 |
| Wintergreen | 0.029 | 1.2 |
| Glycerol | 0.065 | 2.7 |
| Zinc Gluconate | 0.05 | 2.1 |
| Peppermint oil | 0.51 | 21.2 |
| xylitol | 0.16 | 6.6 |
| stannous fluoride | 0.001 | 0.04 |
| Total lozenge | 2.408 | 100.0 |
| weight | | |

The lozenges were prepared according to the above (e.g., see Example I) by mixing the above components at room temperature and ambient humidity. The mixture of the components resulted in a soft, wet composition that was pressed into lozenge forms of about 0.42 g each. In the oral environment with multiple human test subjects, the lozenges dissolved by dissolution in about 1 hour, the exact time depending on the extent of movement of the lozenge in the mouth of the various subjects.

### Example VIII

### Vitamin B12 Containing Lozenges

A vitamin B12 lozenge was prepared as a means to administer vitamin B12 through direct absorption through the oral mucosa. Lozenges were prepared according to Table VII below by mixing the referenced components at room temperature and ambient humidity (e.g., in the manner described in Example I). The mixture of the components resulted in a soft, wet composition that was pressed into lozenge forms of about 0.42 g each. In the oral environment the lozenges dissolved by dissolution in about 1 hour, depending on the extent of movement of the lozenge in the mouth of the various subjects.

TABLE VII

| | Grams | % |
|---|---|---|
| Ethylcellulose (e.g. | 0.4 | 29.4 |
| ethocel 100) | | |
| Sucralose | 0.12 | 8.8 |
| Gum Arabic | 0.2 | 14.7 |
| Vitamin B12 | 0.022 | 1.6 |
| Steric Acid | 0.05 | 3.7 |
| Glycerol | 0.05 | 3.7 |
| Peppermint oil | 0.42 | 30.8 |
| xylitol | 0.1 | 7.3 |
| Total lozenge | 1.362 | 100.0 |
| weight | | |

### Example IX

### Coenzyme Q 10 (CoQ10) Containing Lozenges

Coenzyme Q10 has been reported to enhance the health of the gum, and thereby decreasing the risk of periodontal disease as well as treatment of the disease. The following formulation was prepared:

TABLE VIII

| | Grams | % |
|---|---|---|
| Ethylcellulose (e.g. | 0.4 | 20.3 |
| ethocel 100) | | |
| Gum Arabic | 0.1 | 5.1 |
| CoQ10 mix | 0.4 | 20.3 |
| Stevia | 0.08 | 4.1 |
| Magasweet | 0.1 | 5.1 |
| Wintergreen | 0.3 | 15.2 |
| Glycerol | 0.04 | 2.0 |
| Steric Acid | 0.05 | 2.5 |
| Xylitol | 0.5 | 25.4 |
| Total lozenge | 1.97 | 100.0 |
| weight | | |

Lozenges were prepared according to the above Table VIII (e.g., in accordance with Example I) by mixing the above components at room temperature and ambient humidity. The mixture of the components resulted in a soft, wet composition that was pressed into lozenge forms of about 0.42 g each. In the oral environment the lozenges dissolved by dissolution in about 1 hour, depending on the extent of movement of the lozenge in the mouth of the various subjects.

### Example X

### OHL Formulation

Lozenges were prepared by mixing 0.124 g (25.8%) ethylcellulose, such as ETHOCEL® Standard 100 Premium; 0.02 g (4.2%) wintergreen and 0.087 g (18.1%) peppermint oil; 0.093 g (19.3%) gum arabic; 0.01 g (2.1%) zinc gluconate; 0.032 g (6.7%) sucralose; and 0.032 g (6.7%) xylitol together along with other additives (see Table IX, below) at room temperature and ambient humidity. Admixture of the components resulted in a soft, wet composition that was formed into a lozenge via a press and allowed to set for 24 hours. Then, lozenges each weighing 0.2 g were cut.

In the oral environment of a human test subject, after 2 hours in the mouth, the lozenges entirely dissolved thereby releasing the essential oil component, zinc, and the other components into the aqueous environment of the oral cavity.

The constituents of the dosage form included:

TABLE IX

| | Grams | % |
|---|---|---|
| Ethylcellulose | 0.124 | 25.8 |
| Sucralose | 0.032 | 6.7 |
| Wintergreen | 0.02 | 4.2 |
| Sodium bicarbonate | 0.013 | 2.7 |
| Eucalyptol | 0.0048 | 1.0 |
| Thymol | 0.0048 | 1.0 |
| Glycerol | 0.013 | 2.7 |
| Zinc Gluconate | 0.01 | 2.1 |
| Gum arabic | 0.093 | 19.3 |
| Peppermint oil | 0.087 | 18.1 |
| Xylitol | 0.032 | 6.7 |
| Menthol | 0.047 | 9.8 |
| Total lozenge weight | 0.4806 | 100 |

All publications and patents cited in this specification are herein incorporated by reference as if each individual publication or patent were specifically and individually indicated to be incorporated by reference.

While the invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the invention. All such modifications are intended to be within the scope of the claims appended hereto.

What is claimed is:

1. A sustained release lozenge, comprising a gradually dissolving matrix, wherein the matrix comprises:
(a) an essential oil component, and
(b) a water-insoluble polymer;
wherein, in an aqueous environment, the matrix substantially dissolves, over a prolonged period of time.

2. The sustained release lozenge according to claim 1, wherein the essential oil component comprises an essential oil, a constituent of an essential oil, or a mixture thereof.

3. The sustained release lozenge of claim 2, wherein the lozenge comprises an essential oil comprising an oil selected from the group consisting of: a citrus oil, lemon oil, lime oil, neroli oil, orange oil, a mint oil, peppermint oil, spearmint oil, anise oil, cardamom oil, cinnamon oil, clove oil, coriander oil, eucalyptus oil, fennel oil, lemongrass oil, nutmeg oil, eriodictyon fluid extract, glycyrrhiza extract, and combinations thereof.

4. The sustained release lozenge of claim 2, wherein the lozenge comprises a constituent of an essential oil comprising a terpene, a sesquiterpene, or combinations thereof.

5. The sustained release lozenge of claim 1, wherein the water-insoluble polymer comprises ethylcellulose.

6. The sustained release lozenge according to claim 5, wherein the ethylcellulose comprises a solution viscosity in the range of about 41 cP to 110 cP.

7. The sustained release lozenge according to claim 6, wherein the ethylcellulose comprises a solution viscosity in the range of about 90 cP to 110 cP.

8. The sustained release lozenge of claim 1, wherein the water-insoluble polymer comprises a substantially uniform particle size.

9. The sustained release lozenge of claim 1, wherein the water-insoluble polymer comprises an average particle size diameter in the range of about 1 micron to about 250 microns.

10. The sustained release lozenge of claim 1, wherein the water-insoluble polymer dissolves entirely over a prolonged period of time.

11. The sustained release lozenge of claim 1, wherein the water-insoluble polymer dissolves without breaking down into pieces.

12. The sustained release lozenge of claim 1, wherein the water-insoluble polymer comprises a solubility in water of less than 5 weight %.

13. The sustained release lozenge of claim 1, wherein the weight ratio of the water-insoluble polymer to essential oil component is in the range of about 2:1 to 4:1.

14. The sustained release lozenge of claim 1, wherein the combined weight % of the water-insoluble polymer and the essential oil component comprises from about 25 to about 100% weight % of the lozenge.

15. The sustained release lozenge of claim 1, wherein the dissolution of the lozenge occurs over a period of about 15 minutes to about 6 hours.

16. The sustained release lozenge of claim 1, wherein the dissolution of the lozenge results in the gradual release of the essential oil component.

17. The sustained release lozenge of claim 1, further comprising a water-soluble agent.

18. The sustained release lozenge of claim 17, wherein the water-soluble agent comprises a solubility in water that is greater than or equal to 5 weight %.

19. The sustained release lozenge of claim 17, wherein the water-soluble agent comprises a substantially uniform particle size.

20. The sustained release lozenge of claim 17, wherein the weight ratio of the water-insoluble polymer to water-soluble agent is in the range of about 2:1 to 5:1.

21. The sustained release lozenge of claim 17, wherein the water-soluble agent comprises gum arabic.

22. The sustained release lozenge of claim 21, wherein the gum arabic comprises an average particle size diameter in the range of about 1 micron to about 250 microns.

23. The sustained release lozenge of claim 1, further comprising an effective sweetening amount of a sweetener selected from a sugar, a non-sugar sweetening agent, and a mixture thereof.

24. The sustained release lozenge of claim 1, further comprising an effective amount of a beneficial agent.

25. The sustained release lozenge of claim 21, wherein the beneficial agent is a member of the group consisting of: a saliva substitute, an agent for treating the common cold, a local antibiotic, a local anesthetic agent, pilocarpine, vitamin C, a source of Zn2+, chloride, propionate, butyrate, n-butyrate, β-hydroxybutyrate, benzoate, formate, sulfate, a diet aid, 5-hydroxytryptophan, tyrosine, phenylalanine, pseudoephedrine, ephedrine, phenylpropanolamine, chromium picolinate, aspirin, caffeine, nicotine, a herbal mixture or extract thereof, guarana and ma huang, a source of fluoride ion, and combinations thereof.
